(19) United States
(12) Patent Application Publication
(10) Pub. No.: US 2023/0152228 A1
(43) Pub. Date: May 18, 2023

(54) SYSTEM, METHOD AND OPTICAL MODULE FOR MULTI-FIBER PHOTOMETRY WITH PATTERNED SAMPLE STIMULATION

(71) Applicant: Optomak, Inc., Quebec (CA)

(72) Inventors: Alex Côté, Quebec (CA); Jean-Luc Néron, Quebec (CA); Emir Karamehmedovic, Sarajevo (BA); Sead Doric, L'Ancienne-Lorette (CA)

(21) Appl. No.: 17/530,262

(22) Filed: Nov. 18, 2021

(51) Int. Cl. G01N 21/64; A61B 5/00

(52) U.S. Cl. CPC G01N 21/6456; A61B 5/0071; G01N 2021/6484

(57) ABSTRACT

A optical system and imaging device provide simultaneous or sequential optogenetic stimulation of different locations within a biological sample and fluorescence imaging of the locations. The systems include an illumination array with multiple electrically addressable elements that select a spatial pattern of illumination, an optical fiber bundle with fibers coupled between the array element and target locations at the sample, a fluorescence illumination source, and an imaging system for measuring fluorescence returning from the target locations. The optical imaging devices include a housing and a sample connector configured for receiving a first optical fiber bundle connection including multiple optical fibers. The sample connector has an indexing feature to maintain a fixed relationship between the fibers and internal optical paths of the device. The imaging devices also include an indexed input connector to receive another fiber bundle that carries the stimulus pattern, and a fluorescence excitation input connector.

### BACKGROUND OF THE INVENTION

### 1. Field of the Invention

The present invention relates to multi-fiber photometry and optical sample stimulation, and more particularly concerns a system for in vivo, e.g., in freely-moving/behaving animals and head-fixed animals, or in vitro sample stimulation and fluorescence detection through multiple optical fiber connections with regions of the sample.

### 2. Background of the Invention

Biological fluorescence detection and analysis has been extended to involve optical stimulation of biological samples, which may include operations that modify the samples. Techniques such as optogenetic stimulation, bio-molecular uncaging, and laser-ablation are performed under optical guidance and/or measurement using fluorescence measurements. Systems have been implemented that integrate optical stimulation with fluorescence measurements, such as those systems disclosed in U.S. Pat. Nos. 9,791,683 and 9,846,300. However, while such systems are capable of receiving fluorescence emissions returning from the sample along multiple optical fibers and measuring a response of different regions to the stimulation, the stimulation in such systems is performed across the sample and is typically constrained to the same aperture.

In fluorescence imaging, and more specifically calcium imaging, in order to render a specific population of neurons light sensitive, the population is labeled with genetically encoded functional fluorescent proteins, e.g., calcium indicators, that modulate their fluorescence emission according to the activity of the labeled cells. The labeling enables a visual observation of neuronal activity by observing a fluorescence response to excitation illumination. Typically, the observation is made with a single fiber and the spatial resolution of the measurement is typically limited by the diameter of the fiber optic cable. The collected signal aggregates the activity of hundreds of cells located close to the tip of a fiber optic implant. Early fluorescence imaging systems were developed to record one brain region coupled to one fiber optic cable and typically include light sources (e.g., lamps, LEDs, or lasers), optical fibers, one or more photo-sensors (e.g., photodiodes or image sensors), and a light-filtering assembly that spectrally separates excitation and emission light. Control of the excitation illumination sources and photo-sensors data acquisition are typically managed by a computer. When the samples are in freely behaving animals, an optical rotary joint, such as that described in U.S. Pat. No. 10,564,101, is included to remove twists that may otherwise occur in the fiber optic cable. Entirely optical fiber-based systems have also been proposed, such as those disclosed in P.R.C. Patent No. CN213309653U where spectral separation is performed in fiber bundles.

The above-described fluorescence imaging techniques monitor cell activity by recording temporal fluorescence variations during optical excitation of a fluorescent indicator, e.g., a calcium indicator. In order to simultaneously record the fluorescence activity of multiple brain regions, it has also been proposed to replace the photodiodes with a CMOS image sensor, such as in the systems described in U.S. Patent Application Publication No. 20180228375A1 to image signals from several optical fiber cables that are connected to, and excite different brain regions.

Optogenetics is a neuroscience research technique that uses specific light stimuli for activating or inhibiting cells such as neurons. To render cells light-sensitive, the cells are marked with genetically encoded light sensitive ion channels, e.g., channelrhodopsin (ChR2) for activation of neural activity or halorhodopsin for inhibition of neural activity. Frequently, it is desirable to combine fluorescence imaging with optogenetics in order to gain deeper insight and confirm correlations between specific brain regions activity and behavior of the behaving animal. However, optogenetics and fluorescence imaging do not mix well due to the significant difference between the light intensity requirements, with optogenetics stimulation requiring significantly greater intensity than that required for fluorescence excitation. Excessive optogenetics stimulation or fluorescence excitation illumination may cause photobleaching or trigger phototoxicity mechanisms, and consequently induce damage to fluorescent markers or the targeted tissue. Ideally, optogenetics and fluorescence imaging measurements should be separated spatially, whenever possible, or temporally. Since the optogenetics stimulation illumination is of greater intensity, the stimulation should be confined to the smallest area possible.

For recordings over individual points of interest, fluorescence imaging and optogenetics stimulation have been combined for one or two sites as in the systems disclosed in U.S. Pat. Nos. 9,791,683 and 9,846,300. Additional sites of interest could be added by replicating the systems. However, with larger numbers of recording sites, replication becomes expensive unwieldy. Another approach that has been taken using multiple site fiber photometry and optogenetics uses a single image sensor, one optical fiber bundle, and one light source for each spectral region, significantly reducing the system complexity. However, the above approach lacks specificity, as each site receives the same excitation and optogenetics lights. In some instances, various regions of the sample may be desirably stimulated or altered optically, and those regions may or may not correspond to regions of interest in the fluorescence measurements. It may also be desirable to have control of a number of regions to be stimulated simultaneously or sequentially in a measurement, without stimulating the entire sample.

Therefore, it would be desirable to provide a method and system for performing fluorescence measurements with optical stimulation of a biological sample so that a simultaneous and/or sequential stimulation can be applied to different locations within a sample and that are capable of performing fluorescence measurements in those locations.

### SUMMARY OF THE INVENTION

The objectives of performing optical stimulation and fluorescence measurements in different locations of a biological sample are accomplished in optical systems, optical imaging devices for use in the systems, and methods of operating the systems.

The systems are systems for performing optical stimulation and fluorescence measurements on a sample, and include an illumination array having multiple stimulation illumination elements that are electronically addressable to select a spatial pattern of illumination, and an optical fiber bundle including multiple optical fibers coupled to corresponding ones of the multiple stimulation illumination elements at their proximal ends. The multiple optical fibers are for coupling to target locations within or on a sample at their distal ends, so that activated ones of the multiple stimulation illumination elements provide stimulation at corresponding ones of the target locations. The systems also include a fluorescence illumination source for providing fluorescence illumination and coupled to the proximal end of the optical fiber bundle, and an imaging system coupled to the proximal end of the optical fiber bundle for measuring fluorescence returning from the target locations.

The optical imaging devices for use in the systems include a housing, a sample connector accessible at an exterior of the housing and configured for receiving connection with a first optical fiber bundle including multiple optical fibers. The sample connector has an indexing feature complementary with that of the received connection with the first optical fiber bundle, so that a fixed relationship between the individual ones of the multiple fibers and internal optical paths of the optical imaging device is maintained. The optical imaging devices also include a stimulation illumination input connector accessible at the exterior of the housing and having a second indexing feature, and the stimulation illumination input connector is configured for receiving connection from a second optical fiber bundle comprising second multiple optical fibers that provide individual elements of a pattern of illumination, so that a fixed relationship between the pattern of illumination and the internal optical paths of the optical imaging device is maintained. The optical imaging devices also include a fluorescence illumination input connector accessible at the exterior of the housing for receiving an optical connection from a fluorescence illumination source, and an output connector accessible at the exterior of the housing for providing fluorescence image information to an external system. The output connector may be an optical connector for providing a fluorescence image to an external optical imaging system, or the device may include an electro-optic sensor array and the output connector may be an electrical connector for connecting the sensor array to an electronic controller.

The summary above is provided for brief explanation and does not restrict the scope of the claims. The description below sets forth example embodiments according to this disclosure. Further embodiments and implementations will be apparent to those having ordinary skill in the art. Persons having ordinary skill in the art will recognize that various equivalent techniques may be applied in lieu of, or in conjunction with, the embodiments discussed below, and all such equivalents are encompassed by the present disclosure.

### BRIEF DESCRIPTION OF THE DRAWINGS

FIG. 1 is a pictorial diagram depicting an example system 10, in accordance with an embodiment of the disclosure.

FIGS. 2A-2D are pictorial diagrams depicting various states of illumination within fiber bundle 14B in example system 10 of FIG. 1, in accordance with embodiments of the disclosure.

FIG. 3 is a block diagram depicting an example system 10A, in accordance with an embodiment of the disclosure and including details of an example optical combiner/splitter device 30A, in accordance with an embodiment of the disclosure.

FIG. 4 is a pictorial diagram depicting details of another example optical combiner/splitter device 30B that may be used in example system 10 of FIG. 1 and example system 10A of FIG. 3, in accordance with an embodiment of the disclosure.

FIG. 5 is a pictorial diagram depicting an example stimulus illumination system 50 that may be used in example system 10 of FIG. 1 and example system 10A of FIG. 3, in accordance with an embodiment of the disclosure.

FIG. 6 is a pictorial diagram depicting yet another stimulus illumination system 60 that may be used in example system 10 of FIG. 1 and example system 10A of FIG. 3, in accordance with an embodiment of the disclosure.

FIG. 7 is a perspective view showing an example multiple fiber optic connector pair that may be used in example system 10 of FIG. 1 and example system 10A of FIG. 3, in accordance with an embodiment of the disclosure.

### DETAILED DESCRIPTION OF ILLUSTRATED EMBODIMENT

The instant disclosure provides optical stimulation and fluorescence photometry systems and methods, along with an optical imaging device that may form part of the systems. The techniques are applicable to optogenetics and fiber photometry fluorescence recording, among other potential applications, both in vivo, e.g., in freely-moving/behaving animals and head-fixed animals, and in in vitro sample analysis. The systems enable optogenetic stimulation, laser ablation and/or biomolecular uncaging as a stimulus to a sample and at different locations within the sample, and are particularly suited to performing simultaneous in vivo fluorescence recording and optogenetic stimulation in biological tissue, for biomedical research applications. The systems and techniques are also applicable in photo-stimulated uncaging of biomolecules or laser ablation and processing. The systems and techniques combine stimulation light and multi-fiber fluorescence photometry recording. The optical stimulation targets selected sites within a predefined grid of multiple sites on the biological sample, while simultaneously enabling photometry recording across some or all of the multiple sites. Fluorescence excitation illumination is provided at different sample locations and fluorescence pattern information is returned from the locations in the sample by introducing the fluorescence excitation illumination across a plurality of fibers forming a fiber bundle, and returning the resulting fluorescence information through the fibers of the fiber bundle from the various locations in the sample, which are collected by imaging the returned fluorescence information across fiber bundle. While the terms “image” and “imaging” are used to describe the fluorescence detection path in the systems disclosed herein, it is understood that the detection of the fluorescence may be performed by a discrete detector per-fiber/location and that the “image” may effectively constitute a single pixel-per fiber (discrete detectors) or the fluorescence information may be detected by a higher-resolution image sensor, such as a CMOS or CCD camera returning a single image in which image regions correspond to the individual optical fibers in the bundle and that may then be processed with an averaging algorithm to produce an intensity value for each optical fiber or location within the sample. The optogenetic stimulation, laser ablation and/or biomolecular uncaging stimulus light is also provided through selected fibers by generating the stimulus light with an illumination array having multiple stimulation illumination elements that are electronically addressable to select a spatial pattern of illumination that is introduced to the fiber bundle. The pattern is not necessarily related to the position of the locations within the sample other than the relationship between the illumination array and the optical fibers, which may be located as needed in order to stimulate the sample in a desired manner. The disclosed device can be used in a wide variety of applications and combinations where delivery of light at different wavelengths to several sites and of various intensity is desired, with the ability to record an optical signal from the several sites.

The combining of the optical stimulation and fluorescence photometry is accomplished using a filtering assembly in which light provided to an optogenetics illumination port from a high-density fiber-optic array that may be arranged in a grid, is collimated and projected on a fiber-optic array at the sample port, which may also be arranged in a grid. Fluorescence excitation is introduced through another connection. Excitation and emission light are spectrally separated and routed by the filter assembly. By inserting light-source-coupled optical fibers in selected receptacles within the grid of an optogenetics illumination port, it is possible to efficiently illuminate the desired targeted sites. The optogenetics stimulation illumination on the biological sample is not necessarily a grid that matches that of the optogenetics illumination port, rather, the arrangement of fibers facing the biological sample can target specific neural centers that are not necessarily in a regular grid, and are known to be at specific positions on the sample. An important aspect of the system is the ability of the system to combine fluorescence excitation light that may target the entire biological sample, with optogenetics stimulation illumination, which is generally of greater intensity than the fluorescence excitation light, and which may target only specific sites on the biological sample while simultaneously recording the fluorescence response.

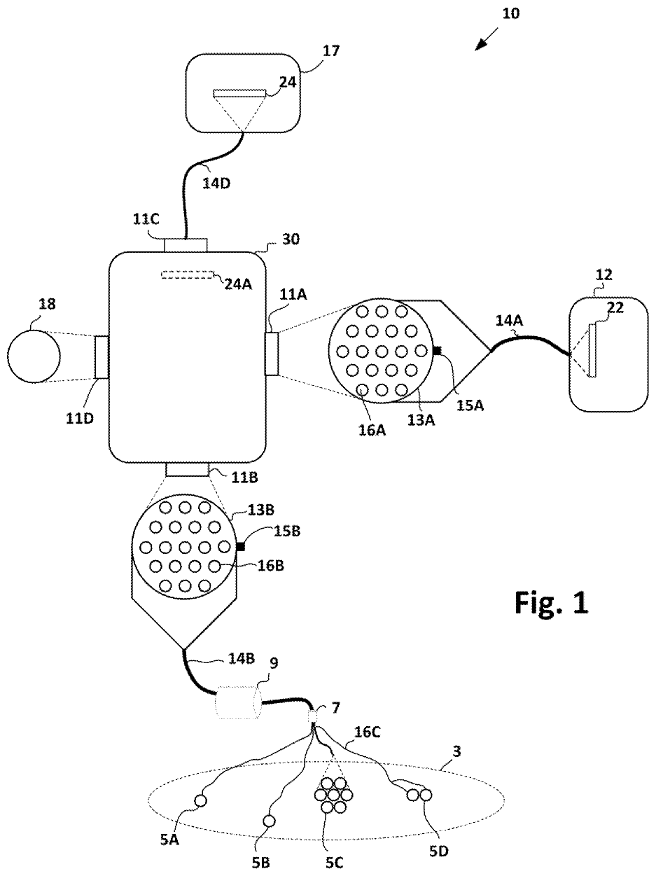

Referring to FIG. 1, a pictorial diagram is shown, depicting an example system 10, in accordance with an embodiment of the disclosure. Example system 10 is an optical measurement system for performing measurements/experiments on a sample 3, which may be, for example, a brain in an in vivo animal specimen or brain tissue forming an ex vivo specimen. Illustrated system 10 is capable of providing stimulus illumination, as described above, that may be for optogenetic stimulus, or may be light for performing uncaging of bio-molecules or for performing laser ablation of tissue within sample 3. For the purpose of simplification of description of system 10 and further systems and devices included in the various Figures, it will be understood that mention of optogenetics with respect to stimulus illumination and measurement will apply also to biomolecular uncaging and laser ablation functions, as well, with selection of suitable power levels and wavelengths understood.

System 10 includes an excitation illumination source 18 for fluorescence measurement/monitoring and a stimulation illumination source 12 for performing stimulus on or in sample 3. In the depicted embodiment, stimulus illumination source 12 includes an illumination array 22 that produces a pattern of illumination that is mapped to individual optical fibers (or alternatively individual sub-bundles of optical fibers) within an optical fiber bundle 14A. Alternatively, in any of the locations in which an optical fiber bundle is applied in the disclosure, a multiple core fiber may alternatively be used, with suitable coupling to introduce and receive light to and from the cores in the multiple core fiber. In the illustrated example, a 19-fiber optical fiber bundle 14A is employed to convey stimulation light from individual illumination elements of array 22, which may be an array of light-emitting diodes (LEDs), LED lasers, lamps, or other suitable stimulus illumination source. Sources included in stimulus illumination source 12 can vary in type, number and emitted wavelength, depending on the requirements of the experiment. Each source may be individually controlled by software executing on a controller as described in further detail below. Each of the optical fibers of optical fiber bundle 14A are individually terminated and may be connected to any of the individual sources included in stimulus illumination source 12 as required by the experiment design. Optical fiber bundle 14A is connected to a combiner/splitter device 30 in accordance with an embodiment of the disclosure, with a keyed connector 13A that provides proper optical alignment via a connector key 15A and prevents mis-rotation of optical fiber bundle 14A with respect to a stimulus illumination mating connector 11A provided on a housing of combiner/splitter device 30, so that the patterned relationship between the individual fibers and the illumination source elements is maintained. Connectors 13A and 11A ensure that the transverse and longitudinal position of the ends of the individual fibers are correct, and that efficient coupling of light from the fibers delivering the stimulus illumination to sample 3 is achieved.

In order to provide proper mapping from elements of stimulus illumination array 22 to individual regions 5A-5D within sample 3, i.e. individual cells or groups of cells within sample 3, at which ends of a number of optical fibers 16C are implanted, sample 3 is connected to optical combiner/splitter device 30 with another keyed connector pair: connector 13B, which receives the proximal end of another multiple fiber bundle 14B and has a connector key 15B, and another mating connector 11B provided on the housing or combiner/splitter device 30. The pattern of the stimulus illumination may be generated by using a regular array of optical fibers held together by a mechanical part defining the pattern, i.e., connector 13A. The number, type and length of the optical fibers in the connector can vary to adapt to the experiment. Both the sample connector 13B and the patterned stimulus illumination connector 13A may use the same mechanical design to place and hold the fibers. The number, size and pattern of the optical fibers in the fiber array can vary depending on the requirements of the experiments. Patterns in the sample and optogenetics port can be different as long as there is an overlap and light coupling from optogenetics port to the sample port. As mentioned above, in some embodiments, a multicore fiber may be used instead of an array of single fibers at both the stimulus illumination connector 11A and the sample connector 11B. A tapered multicore fiber connector or a similar device is used to couple light to/from the different cores of the multicore fiber at the optogenetics port. Multicore fiber is more compact and can be easier to handle than a fiber array.

Also illustrated are a multiple-channel optical rotary joint 9, which may be needed for live animal studies and a multi-fiber cannula 7 (or alternatively, multiple cannulas), which may provide detachable connections to sample 3. Details of multi-channel rotary joints that may be used in implementing example system 10 are disclosed in U.S. Pat. No. 9,046,659, the disclosure of which is incorporated herein by reference. Regions 5A-5D are illustrations of various combinations of optical fibers that show other than 1:1 correspondence between regions and physical location. For example, a high-stimulation intensity region (or a higher resolution fluorescence image measurement region) 5C might be implemented using, for example, 7 fibers out of multiple fiber bundle 14B as shown, while regions 5A and 5B are connected to only one fiber and region 5D is connected to two. In addition to allowing for disparate regions at which individual optical fibers 16C terminate optical fiber bundle 14B, because stimulus illumination array 22 is electronically controlled and composed of individual illumination elements, stimulus illumination can be sequentially applied to regions 5A-5D, by addressing the individual elements of stimulus illumination array 22, or any combination of regions 5A-5D may be simultaneously stimulated by stimulus light. In general, a one-to-one mapping of elements in stimulus illumination array 22 to a corresponding region is not required, as long as a specific element in stimulus illumination array 22 is coupled to a specific region of sample 3, for those elements of stimulus illumination array 22 that are to be used in an experiment. In some embodiments, two or more separate patterned stimulation illumination ports, e.g., for optogenetic stimulation, may be provided. For example, one port may be provided for each function among multiple functions that stimulate sample 3 at different wavelengths, such as wavelengths for neuron activation or inhibition. In such embodiments, additional filters, e.g., dichroic mirrors are needed to combine light from additional input port(s) into the sample connection. Patterns of any of the stimulation illumination input ports would then coincide fully or partially with the pattern of stimulus illumination provided at the sample port.

The fluorescence measurement/monitoring function of system 10 is provided by a fluorescence excitation illumination source 18 that is coupled to optical combiner/splitter device 30 at a single-fiber (or fiber lightguide) connector 11D that receives the fluorescence illumination from fluorescence excitation illumination source 18 and produces substantially uniform illumination across the proximal ends of fiber bundle 14B at connector 11B, via action of optical combiner/splitter device 30, details of which will be described in further detail below. Alternatively, fluorescence excitation illumination source 18 may be connected directly to optical combiner/splitter device 30. Multiple excitation illumination sources 18 having differing wavelengths may be coupled with individual optical fibers to multiple connectors 11B to perform detection of different markers having different excitation wavelengths. While the embodiments disclosed herein are described with respect to fluorescence excitation, it is understood that photoluminescence stimulation generally, including fluorescence and up-conversion are contemplated for measurement using the devices and systems disclosed herein. Excitation illumination source 18 may be or include a pulsed laser for energy resonance transfer measurements/decay time measurements and may comprise one or more LED sources that are combined into a single optical fiber. The power level and activation times of the individual sources are controlled electronically by a controller or computer under software control as described in further detail below. Fluorescence measurement/monitoring of the fluorescence light returning from sample 3 through optical fiber bundle 14B is made via an image sensor 24 within a fluorescence detector 17 that is coupled to optical combiner/splitter device 30 by an optical fiber cable (or fiber lightguide) 14D at a connector 11C. Alternatively, an image sensor 24A may be integrated within optical combiner/splitter device 30, in which case an electrical control and image data interface is provided at the exterior of optical combiner/splitter device 30, rather than optical fiber cable 14D. Image sensor 24 and 24A, may be CMOS or CCD arrays, or may be an array of photodiodes or photomultiplier tubes (PMTs) if faster acquisition time is needed. Fluorescence detector 17 may be, or may include a spectrometer that measures a wavelength of the fluorescence emissions returning from sample 3.

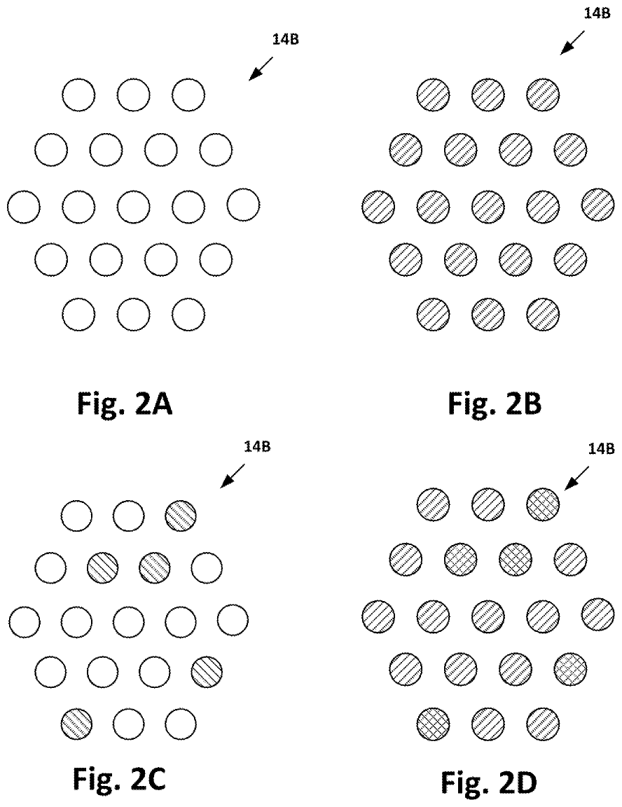

Referring now to FIGS. 2A-2D, pictorial diagrams depict various states of illumination within fiber bundle 14B in example system 10 of FIG. 1, in accordance with embodiments of the disclosure. FIG. 2A shows a state in which all of the stimulus illumination elements in array 22 of FIG. 1 are off, and no optogenetic stimulus or fluorescence excitation light is provided through optical fiber bundle 14B. FIG. 2B illustrates a state in which fluorescence excitation, shown as an upward-right hashing, is provided to optical fiber bundle 14B, which is substantially uniform across the entire set of fibers. FIG. 2C illustrates stimulus illumination provided to only some of the optical fibers of optical fiber bundle 14B, as illustrated by a downward-right hashing. FIG. 2D illustrates simultaneous application of the fluorescence illumination of FIG. 2B and the stimulus illumination of FIG. 2C.

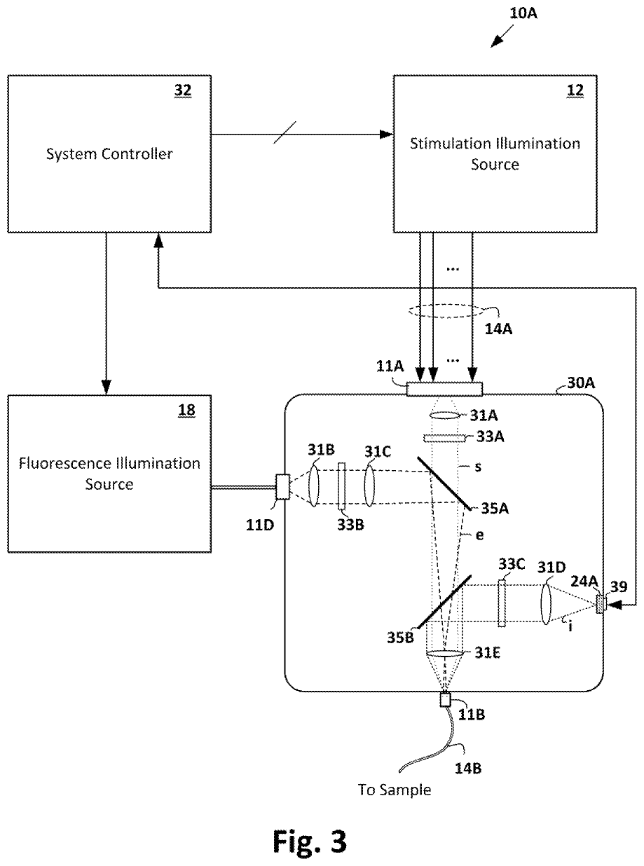

Referring now to FIG. 3, a block diagram depicts an example system 10A, in accordance with an embodiment of the disclosure. Example system 10A is illustrated to show control of systems such as example system 10, and details of an example combiner/splitter device 30A that may be used to implement optical combiner/splitter device 30 in example system 10 of FIG. 1. Therefore, example system 10A is similar to, and has common elements with, example system 10, so only differences between example system 10A and example system 10 will be described in detail below. A system controller 32 provides power to and control of fluorescence illumination source 18 so that fluorescence measurements can enabled by timed application of fluorescence excitation illumination. System controller 32 also controls stimulation illumination source 12, to select whether or not stimulus illumination is provided to optical fiber bundle 14A, and if so, which fibers within optical fiber bundle 14A will receive stimulus illumination, by selecting which elements in stimulus illumination array 22 of FIG. 1 are active. Optical combiner/splitter device 30A includes a stimulation illumination optical path s containing the selected pattern of stimulus illumination, a fluorescence excitation optical path e, which carries the single-spot fluorescence excitation illumination, and an imaging optical path i, along with the image of returning fluorescence is guided to optical combiner/splitter device 30A to image sensor 24A. A power, control and data interface 39 is provided at the exterior of optical combiner/splitter device 30A to interface image sensor 24A with system controller 32, so that system controller 32 can provide fluorescence detection data output for further processing/analysis.

Within example optical combiner/splitter device 30A, the combining and separation of light at different wavelengths is performed by a pair of dichroic mirrors 35A, 35B. Connector 11B receives an interchangeable pattern of fibers forming an array within optical fiber bundle 14B that can be inserted into the sample port connector 11B, which positions the patterned fiber optic array longitudinally at the focal plane of an infinity corrected optical lens 31E. Connector 11D receives the optical fiber connection from fluorescence illumination source 18. The fluorescence excitation light is received by a pair of optical lenses 31B, 31C, that, together with infinity corrected optical lens 31E project uniform illumination at the proximal end of optical fiber bundle 14B using the Kohler illumination method. The spot-size of the uniform illumination arriving at the sample port connector 11B is larger than the receptacle having the fiber array arranged in the pattern at the sample port, and thus couples to all fibers in all possible fiber array patterns. A band-pass filter 33B is used to select only the wavelength range of the excitation light received at connector 11D that is needed to generate the fluorescent signal. In some embodiments, the fluorescence excitation illumination may be frequency modulated, in order to produce a signal modulated at the same frequency. Such modulation techniques enable use of lock-in techniques to isolate and detect the signal with a greatly improved signal-to-noise ratio (SNR) in the electrical domain. In some embodiments, the fluorescence excitation illumination may lie in the infrared (IR) or near-infrared (NIR) region. The fluorescence excitation illumination may thereby penetrate deeper into tissue and may be used to excite unconverting nanoparticles (UCNPs), as disclosed in U.S. Pat. No. 9,522,288, which then emit light in the ultraviolet (UV) to visible (VIS) region of the spectrum. Excitation of UCNPs usually requires higher light intensity than does excitation of fluorophores. Therefore, such excitation may not necessarily be projected evenly into all optical fibers of the fiber array in the sample port, but instead may target one or a selected group of fibers in the sample port.

A dichroic mirror 35B separates the returning fluorescence pattern, i.e., the fluorescence signal or resultant image, from the illumination pathways so that the light returning from sample 3 via sample connector 11B is directed toward image sensor 24A, which, in the depicted embodiment is a CMOS sensor array. Image sensor 24A is at the focal point of an infinity corrected imaging lens 31D. In some embodiments, the image of the signal may be inverted or magnified by the imaging optics. A band-pass filter 33C selects the wavelength range of the fluorescence signal. The pattern of the multiple fibers of fiber bundle 14B connected to sample connector 11B is imaged on image sensor 24A and the signal of each individual optical fiber within the pattern can be distinguished. The data from the imaging sensor is transferred to system controller 32, and processed by image processing software. A dichroic mirror 35A combines the stimulus light pattern received from optical fiber bundle 14A at connector 11A, which is at the focal point of an infinity-corrected optical lens 31A. A bandpass filter 33A selects the wavelength range needed to stimulate sample 3. In other embodiments, dichroic mirrors 35A, 35B may be replaced with other types of combiners/splitters. For example, combining of excitation illumination from fluorescence illumination source 18 with the stimulation illumination received from connector 11A may be performed by a spectral combiner, a polarization combiner, or by a beam splitter.

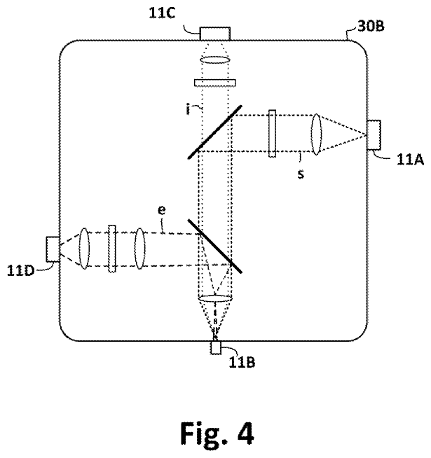

Referring now to FIG. 4, a pictorial diagram depicting details of another example optical combiner/splitter device 30B that may be used in example system 10 of FIG. 1 and example system 10A of FIG. 3, is shown, in accordance with an embodiment of the disclosure. Combiner/splitter device 30B is similar to combiner/splitter device 30A of FIG. 3, so only differences between them will be described in detail below. Combiner/splitter device 30B illustrates a different arrangement of components, and it is understood that additional other embodiments and arrangements are possible within combiner/splitter device 30 of FIG. 1. In combiner/splitter device 30B, a dichroic mirror 35C directs the stimulus illumination received from connector 11A via an infinity-corrected optical lens 31G, which is filtered by bandpass filter 33A, to a dichroic mirror 35D which combines the stimulus illumination with the fluorescence excitation illumination received at connector 11C. Dichroic mirror 35C separates the fluorescence signal, i.e., the fluorescent image returning from sample connector 11B and that is passed through dichroic mirror 35D. Bandpass filter 33C selects the wavelength range of the fluorescence signal, which is coupled to an external imaging system via connector 11C, which is provided with the image through an infinity-corrected lens 31F.

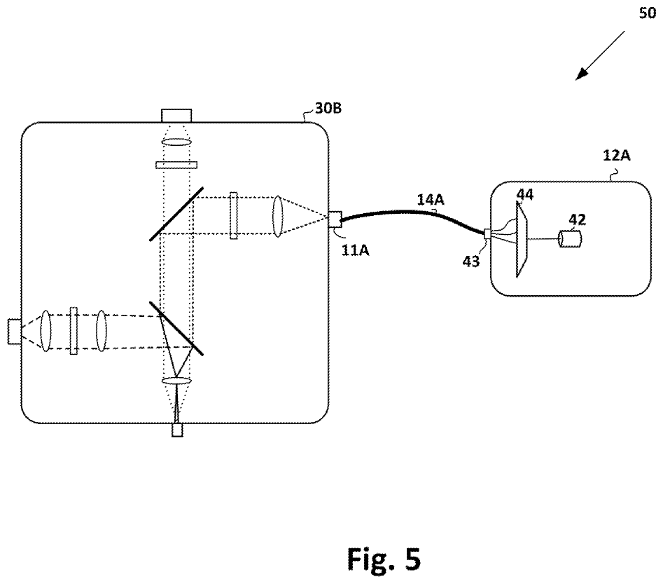

Referring now to FIG. 5, a pictorial diagram depicts an example stimulus illumination system 50 that may be used in example system 10 of FIG. 1 and example system 10A of FIG. 3, in accordance with an embodiment of the disclosure. The depicted embodiment illustrates a use of optical combiner/splitter device 30B that does not require a patterned stimulus array or other arrangement of stimulus sources, and which applies to other optical combiner splitter devices disclosed herein. Stimulus illumination is provided by a stimulus illumination source 12A that includes a source element 42, e.g., an LED or laser diode, etc., as described above. The output of source element 42 is demultiplexed by an optical switch that selects one of several optical fiber connections provided to a connector 43 that receives an end of optical fiber bundle 14A. The depicted embodiment allows for sequential selection of individual ones of optical fibers 16C in the system of FIG. 1, to illuminate a single region of sample 3.

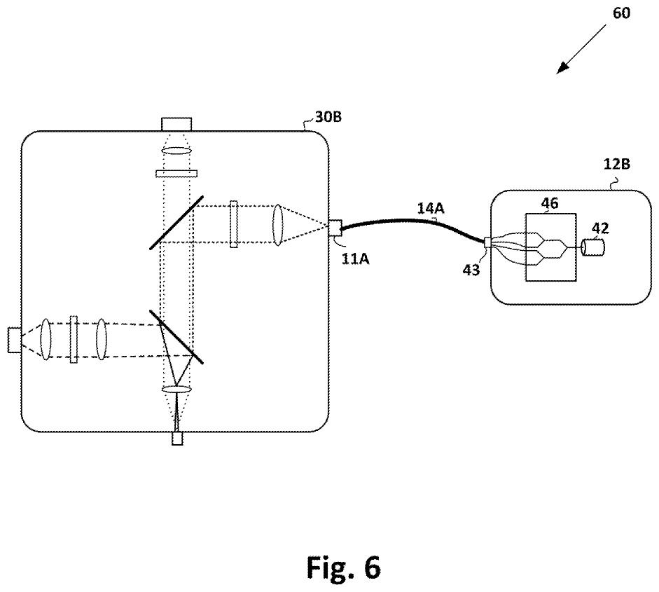

Referring now to FIG. 6, a pictorial diagram depicts an example stimulus illumination system 60 that may be used in example system 10 of FIG. 1 and example system 10A of FIG. 3, in accordance with an embodiment of the disclosure. The depicted embodiment illustrates a use of optical combiner/splitter device 30B that does not require a patterned stimulus array or other arrangement of stimulus sources, and which applies to other optical combiner splitter devices disclosed herein. Stimulus illumination is provided by a stimulus illumination source 12B that includes source element 42 and a beam splitter 46 that divides the stimulus illumination among several optical fiber connections provided to connector 43. The depicted embodiment allows for simultaneous stimulus illumination provided to individual optical fibers 16C in the system of FIG. 1, to illuminate multiple regions of sample 3 simultaneously.

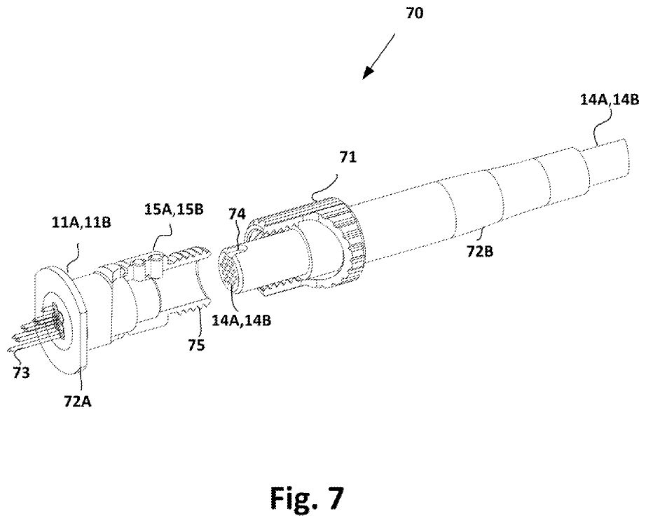

FIG. 7 is a perspective view showing an example multiple fiber optic connector pair 70 that may be used in example system 10 of FIG. 1 and example system 10A of FIG. 3, in accordance with an embodiment of the disclosure. A plurality of polished ends of optical fibers 73 are inserted in a “chassis-type” multiple-position connector 11A,11B, which are then brought into proximity with polished ends of optical fiber bundle 14A, 14B that provide the pattern that is conveyed through the multiple fiber optic connector pair 70 as described above for the stimulus illumination and sample connections. Connector keys 15A, 15B provide a rotationally fixed indexing system that rigidly holds connector 72A in a fixed rotational position by insertion in mating index groove 74 of connector 72B. A threaded portion 75 of connector 72A receives a threaded knurled sheath 71 that secures 72B to connector 72A.

In summary, this disclosure shows and describes systems and methods for performing optical stimulation and fluorescence measurements on a sample. The methods are methods of operation of the systems. The systems may include an illumination array having multiple stimulation illumination elements that are electronically addressable to select a spatial pattern of illumination, an optical fiber bundle including multiple optical fibers coupled to corresponding ones of the multiple stimulation illumination elements at a proximal end of the optical fiber bundle and for coupling to target locations within or on a sample at a distal end of the optical fiber bundle, so that activated ones of the multiple stimulation illumination elements provide stimulation at corresponding ones of the target locations. The systems may also include a fluorescence illumination source for providing fluorescence illumination coupled to the proximal end of the optical fiber bundle, and an imaging system coupled to the proximal end of the optical fiber bundle for measuring fluorescence returning from the target locations.

In some example embodiments, the system may include a combiner as an optical device integrated in the system and located at the proximal end of the optical fiber bundle. The combiner may have a first input coupled to the illumination array, a second input coupled to the fluorescence illumination source and an output coupled to the imaging system. In some example embodiments, the optical fiber bundle includes a first connector at the proximal end, and the first connector may have at least one first indexing feature having a fixed spatial relationship with individual ones of the multiple optical fibers. The combiner may further include a housing, a sample connector accessible at an exterior of the housing for receiving connection of the first connector, the sample connector may have at least one first complementary indexing feature, so that a fixed correspondence between the individual ones of the multiple fibers and the multiple stimulation elements is maintained. The optical fiber bundle may be a first optical fiber bundle, the combiner may further include a stimulation illumination input connector accessible at an exterior of the housing and having a second indexing feature, and the system may further include a second optical fiber bundle comprising second multiple optical fibers coupled to corresponding elements of the illumination array at a distal end thereof and coupled to the stimulation illumination input connector by a second connector. The second connector may have at least one second complementary indexing feature complementary to the second indexing feature, so that a fixed correspondence between the individual ones of the second multiple fibers and the elements of the illumination array is maintained.

In some example embodiments, the combiner may further include a fluorescence illumination input connector accessible at an exterior of the housing for receiving an optical connection from the fluorescence illumination source, and an output connector for receiving a connection conveying fluorescence image information to the imaging system. In some example embodiments, the output connector is an electrical connector, and the imaging system may include an opto-electronic image sensor mounted inside the housing and having data and control signals coupled to the output connector, so that the opto-electronic image sensor receives an image of fluorescence returning from the multiple optical fibers at the sample connector. In some example embodiments, the output connector may be an optical connector for receiving a connection to an external optical connection to convey an image of fluorescence returning from the multiple optical fibers at the sample connector to an image sensor of the imaging system. In some example embodiments, the fluorescence illumination source provides a spot size larger than the optical aperture of the optical fiber bundle at the sample connector, so that the fluorescence illumination is coupled substantially uniformly to the multiple optical fibers at the sample connector.

In some example embodiments, the combiner further includes a first lens system for imaging the spatial pattern of illumination received from the stimulation illumination input connector on the sample connector, and a second lens system for imaging the light returning from the sample through the first dichroic plate at the output connector. In some example embodiments, the system includes an electronic controller coupled to the illumination array for controlling provision of illumination from one or more of the multiple stimulation illumination elements to select the spatial pattern of illumination. The illumination array may include one or more optical switches coupled to the electronic controller for controlling the provision of illumination from the multiple stimulation illumination elements to select the spatial pattern of illumination, whereby the multiple stimulation illumination elements may remain active and selectable via the one or more optical switches to provide or not provide a contribution to the spatial pattern of illumination.

It should be understood, especially by those having ordinary skill in the art with the benefit of this disclosure, that the various operations described herein, particularly in connection with the figures, may be implemented by other components. The order in which each operation of a given method is performed may be changed, and various elements of the systems illustrated herein may be added, reordered, combined, omitted, modified, etc. It is intended that this disclosure embrace all such modifications and changes and, accordingly, the above description should be regarded in an illustrative rather than a restrictive sense. Similarly, although this disclosure makes reference to specific embodiments, certain modifications and changes may be made to those embodiments without departing from the scope and coverage of this disclosure. Moreover, any benefits, advantages, or solutions to problems that are described herein with regard to specific embodiments are not intended to be construed as a critical, required, or essential feature or element.

While the disclosure has shown and described particular embodiments of the techniques disclosed herein, it will be understood by those skilled in the art that the foregoing and other changes in form, and details may be made therein without departing from the spirit and scope of the disclosure.

What is claimed:

1. A system for performing optical stimulation and fluorescence measurements on a sample, the system comprising:
an illumination array comprising multiple stimulation illumination elements that are electronically addressable to select a spatial pattern of illumination;
an optical fiber bundle comprising multiple optical fibers coupled to corresponding ones of the multiple stimulation illumination elements at a proximal end thereof and for coupling to target locations within or on a sample at a distal end thereof, whereby activated ones of the multiple stimulation illumination elements provide stimulation at corresponding ones of the target locations;
a fluorescence illumination source for providing fluorescence illumination coupled to the proximal end of the optical fiber bundle; and
an imaging system coupled to the proximal end of the optical fiber bundle for measuring fluorescence returning from the target locations.

2. The system of claim 1, further comprising a combiner located at the proximal end of the optical fiber bundle, the combiner having a first input coupled to the illumination array, a second input coupled to the fluorescence illumination source and an output coupled to the imaging system.

3. The system of claim 2, wherein the optical fiber bundle includes a first connector at the proximal end thereof, wherein the first connector has at least one first indexing feature having a fixed spatial relationship with individual ones of the multiple optical fibers, and wherein the combiner further comprises:
a housing; and
a sample connector accessible at an exterior of the housing for receiving connection of the first connector, wherein the sample connector has at least one first complementary indexing feature, so that a fixed correspondence between the individual ones of the multiple fibers and the multiple stimulation elements is maintained.

4. The system of claim 3, wherein the optical fiber bundle is a first optical fiber bundle, wherein the combiner further comprises a stimulation illumination input connector accessible at the exterior of the housing and having a second indexing feature, wherein the system further comprises a second optical fiber bundle comprising second multiple optical fibers coupled to corresponding elements of the illumination array at a distal end thereof and coupled to the stimulation illumination input connector by a second connector, wherein the second connector has at least one second complementary indexing feature complementary to the second indexing feature, so that a fixed correspondence between the individual ones of the second multiple fibers and the elements of the illumination array is maintained.

5. The system of claim 4, wherein the combiner further comprises:
a fluorescence illumination input connector accessible at the exterior of the housing for receiving an optical connection from the fluorescence illumination source; and
an output connector for receiving a connection conveying fluorescence image information to the imaging system.

6. The system of claim 5, wherein the output connector is an electrical connector, wherein the imaging system includes an opto-electronic image sensor mounted inside the housing and having data and control signals coupled to the output connector, wherein the opto-electronic image sensor receives an image of fluorescence returning from the multiple optical fibers at the sample connector.

7. The system of claim 5, wherein the output connector is an optical connector for receiving a connection to an external optical connection to convey an image of fluorescence returning from the multiple optical fibers at the sample connector to an image sensor of the imaging system.

8. The system of claim 5, wherein the fluorescence illumination source provides a spot size larger than the optical aperture of the optical fiber bundle at the sample connector, so that the fluorescence illumination is coupled substantially uniformly to the multiple optical fibers at the sample connector.

9. The system of claim 4, wherein the combiner further comprises:
a first lens system for imaging the spatial pattern of illumination received from the stimulation illumination input connector on the sample connector; and
a second lens system for imaging the light returning from the sample through the first dichroic plate at the output connector.

10. The system of claim 1, further comprising an electronic controller coupled to the illumination array for controlling provision of illumination from one or more of the multiple stimulation illumination elements to select the spatial pattern of illumination.

11. The system of claim 10, wherein the illumination array comprises one or more optical switches coupled to the electronic controller for controlling the provision of illumination from the multiple stimulation illumination elements to select the spatial pattern of illumination, whereby the multiple stimulation illumination elements may remain active and selectable via the one or more optical switches to provide or not provide a contribution to the spatial pattern of illumination.

12. An optical imaging device, comprising:
a housing; and
a sample connector accessible at an exterior of the housing and configured for receiving connection with a first optical fiber bundle comprising multiple optical fibers, wherein the sample connector has an indexing feature complementary with that of the received connection with the first optical fiber bundle, so that a fixed relationship between the individual ones of the multiple fibers and internal optical paths of the optical imaging device is maintained;
a stimulation illumination input connector accessible at the exterior of the housing and having a second indexing feature, wherein the stimulation illumination input connector is configured for receiving connection from a second optical fiber bundle comprising second multiple optical fibers that provide individual elements of a pattern of illumination, so that a fixed relationship between the pattern of illumination and the internal optical paths of the optical imaging device is maintained;
a fluorescence illumination input connector accessible at the exterior of the housing for receiving an optical connection from a fluorescence illumination source; and
an output connector accessible at the exterior of the housing for providing fluorescence image information to an external system.

13. The optical imaging device of claim 12, wherein the output connector is an electrical connector, wherein the optical imaging device includes an opto-electronic image sensor mounted inside the housing and having data and control signals coupled to the output connector, wherein the opto-electronic image sensor receives an image of fluorescence returning from the multiple optical fibers at the sample connector.

14. The optical imaging device of claim 12, wherein the output connector is an optical connector for receiving a connection to an external optical connection to convey an image of fluorescence returning from the multiple optical fibers at the sample connector.

15. The optical imaging device of claim 14, further comprising:
at least one first optical splitter/combiner positioned on a first optical path between the sample connector and the output connector for directing the spatial pattern of illumination from the stimulation illumination input connector to the sample connector; and
at least one second optical splitter/combiner positioned on a second optical path between the sample connector and the first optical splitter/combiner, wherein the at least one second optical splitter/combiner directs the spatial pattern of illumination to the sample connector and directs the fluorescence illumination into the sample connector, and wherein the at least one first optical splitter combiner transmits light returning from the sample that is transmitted through the at least one second optical splitter/combiner to the output connector.

16. A method of performing stimulation and fluorescence measurements on a sample, the method comprising:
selecting a spatial pattern of stimulus illumination from an illumination array comprising multiple stimulation illumination elements by electronically addressing the multiple stimulation illumination elements;
providing the spatial pattern of stimulus illumination to the sample via an optical fiber bundle comprising multiple optical fibers coupled to corresponding ones of the multiple stimulation illumination elements at a proximal end thereof and coupled to target locations within or on a sample at a distal end thereof, whereby activated ones of the multiple stimulation illumination elements provide stimulation to corresponding ones of the target locations;
providing fluorescence illumination at the proximal end of the optical fiber bundle a from a fluorescence illumination source; and
measuring fluorescence returning from the target locations with an imaging system coupled to the proximal end of the optical fiber bundle.

17. The method of claim 16, wherein at least some of the selected pattern of stimulus illumination provides light for providing optogenetic stimulation at corresponding ones of the target locations.

18. The method of claim 16, wherein at least some of the selected pattern of stimulus illumination provides light for stimulation that uncage caged compounds in corresponding ones of the target locations.

19. The method of claim 16, wherein at least some of the selected pattern of stimulus illumination provides light for stimulation that ablates tissue in corresponding ones of the target locations.

20. The method of claim 16, further comprising:
combining the fluorescence illumination and the spatial pattern of stimulus illumination with an optical combiner having a housing and a sample connector accessible at an exterior of the housing for receiving connection of the optical fiber bundle;
providing the combined fluorescence illumination and the spatial pattern of stimulus illumination to the sample connector; and
preventing mis-rotation of the optical fiber bundle with respect to the sample connector via indexing features of the sample connector and a connector of the optical fiber bundle that mates with the sample connector.

21. The method of claim 20, wherein the optical fiber bundle is a first optical fiber bundle, and wherein the method further comprises:
receiving the spatial pattern of stimulus illumination from a second optical fiber bundle comprising second multiple optical fibers at a stimulation illumination input connector disposed on a second exterior surface of the housing; and
preventing mis-rotation of the spatial pattern of stimulus information with respect to the stimulation illumination input connector via second indexing features of the stimulation illumination input connector and a connector of the second optical fiber bundle, so that a fixed correspondence between the individual ones of the second multiple optical fibers and the elements of the illumination array is maintained.

22. The method of claim 21, further comprising:
receiving an optical connection from the fluorescence illumination source at a fluorescence illumination input connector disposed on a third exterior surface of the housing; and
providing an image output at an output connector for receiving an optical connection to the imaging system.
